(19) United States
(12) Patent Application Publication
(10) Pub. No.: US 2024/0000336 A1
(43) Pub. Date: Jan. 4, 2024

(54) INSERTION SUPPORT SYSTEM, ENDOSCOPE SYSTEM, AND INSERTION SUPPORT METHOD

(71) Applicant: OLYMPUS MEDICAL SYSTEMS CORP., Tokyo (JP)

(72) Inventors: Naoki FUKATSU, Tokyo (JP); Hiromasa FUJITA, Tokyo (JP)

(73) Assignee: OLYMPUS MEDICAL SYSTEMS CORP., Tokyo (JP)

(21) Appl. No.: 18/368,671

(22) Filed: Sep. 15, 2023

Related U.S. Application Data
(63) Continuation of application No. PCT/JP2021/010727, filed on Mar. 17, 2021.

(51) Int. Cl. A61B 5/06; A61B 1/00; A61B 1/31

(52) U.S. Cl. CPC A61B 5/065; A61M 2025/0166; A61B 1/31; A61B 1/00097

(57) ABSTRACT

An insertion support system includes a processor. The processor acquires endoscopy status information including at least one of an endoscopic image, insertion section shape information, and operation recognition information. In the processor, at least one of input pain information and endoscopy status information is input, and pain situation information is acquired upon recognition of a pain situation. The processor acquires examination condition information including at least one of endoscope kind information, patient information, and past examination information, the endoscope kind information being information about a kind of endoscope insertion section used for endoscopy, the patient information being information about patient attributes, and the past examination information being information about past endoscopies. The processor generates insertion support information according to the pain situation information, the endoscopy status information, and the examination condition information.

### CROSS REFERENCE TO RELATED APPLICATIONS

This application is a continuation of International Patent Application No. PCT/JP2021/010727, having an international filing date of Mar. 17, 2021, which designated the United States, the entirety of which is incorporated herein by reference.

### BACKGROUND OF THE INVENTION

International Publication No. 2018/235185 discloses a technique for performing operation guidance in colonoscopy using an endoscope. In this technology, an insertion shape observation device observes the shape of an endoscope insertion section inserted into the large intestine, an endoscope captures an endoscopic image of the large intestine, and an insertion support device determines the insertion status of the endoscope insertion section based on the shape of the endoscope insertion section and the endoscopic image and performs operation guidance of the endoscope based on the determination results.

### SUMMARY OF THE INVENTION

In accordance with one of some aspect, there is provided an insertion support system comprising:
a processor,
wherein the processor is configured to:
acquire endoscopy status information associated with an endoscopy using an endoscope, the endoscopy status information including at least one of an endoscopic image, insertion section shape information, or operation recognition information, the insertion section shape information being information about a shape of an endoscope insertion section, the operation recognition information being information about a change in at least one of the shape or a position of the endoscope insertion section;
receive input of at least one of input pain information or the endoscopy status information, the input pain information being information about a pain of a patient acquired from the patient or a medical worker in real time, and acquire pain situation information upon recognition of a pain situation, the pain situation being a situation where the patient is suffering a pain during the endoscopy;
acquire examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and
generate insertion support information according to the pain situation information, the endoscopy status information, and the examination condition information.

In accordance with one of some aspect, there is provided an endoscope system, comprising:
an endoscope for use in endoscopy; and
a processor,
wherein the processor is configured to:
acquire endoscopy status information associated with the endoscopy using the endoscope, the endoscopy status information including at least one of an endoscopic image, insertion section shape information, or operation recognition information, the insertion section shape information being a shape of an endoscope insertion section, the operation recognition information being information about a change in at least one of the shape or a position of the endoscope insertion section;
receive input of at least one of input pain information or the endoscopy status information, the input pain information being information about a pain of a patient acquired from the patient or a medical worker in real time, and acquire pain situation information upon recognition of a pain situation, the pain situation being a situation where the patient is suffering a pain during the endoscopy;
acquire examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and generate insertion support information according to the pain situation information, the endoscopy status information, and the examination condition information.

In accordance with one of some aspect, there is provided an insertion support method, comprising:
acquiring endoscopy status information associated with an endoscopy using an endoscope, the endoscopy status information including at least one of an endoscopic image, insertion section shape information, or operation recognition information, the insertion section shape information being a shape of an endoscope insertion section, the operation recognition information being information about a change in at least one of the shape or a position of the endoscope insertion section;
receiving input of at least one of input pain information or the endoscopy status information, the input pain information being information about a pain of a patient acquired from the patient or a medical worker in real time, and acquiring pain situation information upon recognition of a pain situation, the pain situation being a situation where the patient is suffering a pain during the endoscopy;
acquiring examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and generating insertion support information according to the pain situation information, the endoscopy status information, and the examination condition information.

### BRIEF DESCRIPTION OF THE DRAWINGS

FIG. 1 is a first configuration example of an insertion support system.

FIG. 2 is a second configuration example of an insertion support system.

FIG. 3 is a configuration example of an endoscope system.

FIG. 4 is a configuration example of an endoscope and an endoscope shape acquisition sensor.

FIG. 5 is an explanatory view of operation of an endoscope.

FIG. 6 is a first detailed configuration example of an insertion support system.

FIG. 7 is an explanatory view of a large intestine site.

FIG. 8 is an example of shape transition of an endoscope insertion section.

FIG. 9 is an explanatory view of pain situation.

FIG. 10 is an explanatory view of pain situation.

FIG. 11 is an explanatory view of pain situation.

FIG. 12 is an explanatory view of pain situation.

FIG. 13 is an explanatory view of pain situation.

FIG. 14 is an explanatory view of pain situation recognition according to a displacement amount.

FIG. 15 is a flowchart of the processing performed by the insertion support system in the first detailed configuration example.

FIG. 16 is a flowchart showing a specific example of recognition of pain situation and generation of insertion support information according to the recognition results.

FIG. 17 is a flowchart showing a specific example of recognition of pain situation.

FIG. 18 is a second detailed configuration example of an insertion support system.

FIG. 19 is a flowchart of the processing performed by the insertion support system in the second detailed configuration example.

FIG. 20 is a third detailed configuration example of an insertion support system.

FIG. 21 is a fourth detailed configuration example of an insertion support system.

FIG. 22 is a fifth detailed configuration example of an insertion support system.

FIG. 23 is an explanatory view of a specific example of recognition of pain situation.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Configuration Example

This section describes an example of a basic configuration of an insertion support system according to the present embodiment. The detailed configuration example of the insertion support system and the correspondence between the detailed configuration example and the basic configuration example thereof are described later.

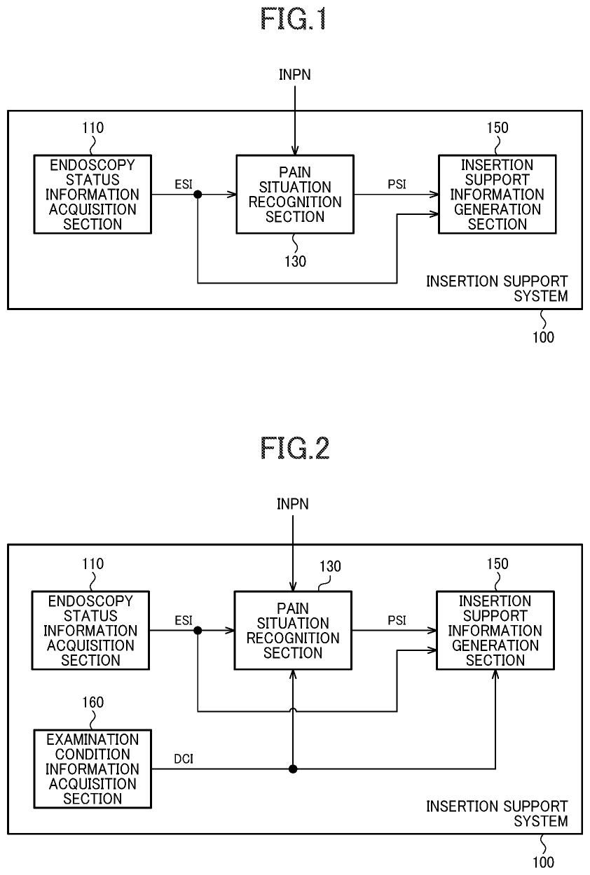

FIG. 1 is a first configuration example of an insertion support system 100. The insertion support system 100 includes an endoscopy status information acquisition section 110, a pain situation recognition section 130, and an insertion support information generation section 150.

The endoscopy status information acquisition section 110 acquires endoscopy status information during endoscopy using an endoscope. The endoscopy status information includes at least one of an endoscopic image, insertion section shape information, and endoscopy status information. The expression “at least one of an endoscopic image, insertion section shape information, and endoscopy status information” refers to the endoscopic image, insertion section shape information, endoscopy status information, or any combination of two or more of them.

The endoscopic image is an image captured by an endoscope. Specifically, the endoscopic image refers to images of individual frames of a moving image captured by an endoscope. The insertion section shape information is information about the shape of the endoscope insertion section, and is acquired, for example, by the insertion section shape observation device, which is described later. The endoscope insertion section is the part of the endoscope that is inserted into the body; in this embodiment, the endoscope insertion section is an insertion section of a flexible endoscope used for digestive tracts, and the like. The operation recognition information is information about a change in at least one of the shape and the position of the endoscope insertion section. That is, the operation recognition information is a time-based change in at least one of the shape and the position of the endoscope insertion section that is made as a result of operation of the endoscope. For example, the insertion section shape observation device observes the shape of the insertion section from time to time to output the insertion section shape information in a time-series manner, and the operation recognition information is acquired from the time-based change of the insertion section shape information thus output in a time-series manner.

The endoscopy status information acquisition section 110 outputs endoscopy status information ESI. This endoscopy status information ESI may be the endoscopy status information itself acquired by the endoscopy status information acquisition section 110 described above, or may be information obtained by processing the acquired endoscopy status information, or a combination thereof.

At least one of the input pain information INPN and the endoscopy status information ESI is input to the pain situation recognition section 130. FIG. 1 shows an example in which both the input pain information INPN and the endoscopy status information ESI are input to the pain situation recognition section 130; however, one of the input pain information INPN and the endoscopy status information ESI may not be input to the pain situation recognition section 130.

The input pain information INPN is information about a pain of a patient acquired from the patient or a medical worker in real time. The patient is a person who is being subjected to endoscopy using an endoscope. The medical worker is a person who can recognize the patient's voice, facial expression, and the like, such as a doctor performing the endoscopy or his/her assistant. The input pain information INPN is acquired, for example, from switches, results of image recognition of facial expressions, results of voice recognition, and the like as described later.

The pain situation recognition section 130 recognizes a pain situation, which is a situation where the patient is suffering a pain during the endoscopy, and acquires pain situation information PSI. The pain situation information PSI is the result of recognition of the pain situation, e.g., the presence or absence of the pain situation, estimated level of the pain, or a combination thereof. The “situation where the patient is suffering a pain” is not limited to the actual occurrence of pain, but can be a situation where the occurrence of pain is assumed. More specifically, the situation where the patient is likely to feel pain is predetermined, and when the situation recognized based on the endoscopy status information ESI matches that predetermined situation, such a situation is recognized as “a situation where the patient is suffering a pain”. Alternatively, when the pain situation is recognized based on the input pain information INPN, the situation is recognized as “a situation where the patient is suffering a pain” when the input pain information INPN indicating the occurrence of pain is input. Also in this case, it is sufficient that the input pain information INPN is information from which the occurrence of pain can be assumed. For example, when the input pain information INPN is input based on the patient's facial expression, it is sufficient that the occurrence of pain is assumed from the facial expression.

The insertion support information generation section 150 generates insertion support information according to the pain situation information PSI and the endoscopy status information ESI. The insertion support information is information that supports the insertion of the endoscope into the patient. Specifically, the insertion support information is information to be presented or notified to the medical worker or insertion/removal device that performs the operation of inserting the endoscope into the patient, and indicates what operation should be performed next for the current insertion status. The insertion support system 100 may, for example, display texts, symbols, images, and the like according to the insertion support information on a display device of the endoscope system, or may output a control signal to the insertion/removal device to cause the insertion/removal device to perform operations or processing indicated by the insertion support information.

During the endoscopy, the patient may suffer a pain in response to the operation of the endoscope insertion section. For example, in the International Publication No. 2018/235185 described above, the pain of the patient being subjected to the endoscopy is not taken into account in the operation guidance; as a result, the operation may be guided regardless of whether the operation causes a pain in the patient or not. Therefore, the patient may suffer a pain because of the operation according to the operation guidance. According to the present embodiment, the insertion support information can be generated in consideration of the situation where the patient is suffering a pain during the endoscopy. Specifically, by performing the operation by a medical worker or an insertion/removal device based on the insertion support information of the present embodiment, when the patient feels pain or before the patient feels pain, it is possible to perform an operation that relieves the patient's pain or an operation that avoids the occurrence of pain beforehand.

When both the input pain information INPN and the endoscopy status information ESI are input, the pain situation recognition section 130 recognizes a pain situation based on the input pain information INPN. The expression “when the input pain information INPN is input” herein means that “when the input pain information INPN indicating occurrence of pain in the patient is input”, i.e., “when a situation where the patient is suffering a pain is recognized based on the input pain information INPN”.

More specifically, when both the input pain information INPN and the endoscopy status information ESI are input, the pain situation recognition section 130 gives priority to the pain situation recognition result based on the input pain information INPN over the pain situation recognition result based on the endoscopy status information ESI. That is, even if the absence of the situation where the patient is suffering a pain is recognized based on the endoscopy status information ESI, if a situation where the patient is suffering a pain is recognized based on the input pain information INPN, the pain situation recognition section 130 outputs pain situation information PSI indicating the situation where the patient is suffering a pain.

More specifically, when the endoscopy status information ESI is input to the pain situation recognition section 130 while the input pain information INPN is not input thereto, the pain situation recognition section 130 recognizes the pain situation based on the endoscopy status information ESI, and the insertion support information generation section 150 generates the insertion support information according to the pain situation recognized based on the endoscopy status information ESI. When the input pain information INPN is input in addition to the endoscopy status information ESI to the pain situation recognition section 130, the pain situation recognition section 130 recognizes the pain situation based on the input pain information INPN, and the insertion support information generation section 150 generates the insertion support information according to the pain situation recognized based on the input pain information INPN.

According to the present embodiment, the pain situation can be recognized based on the input pain information INPN transmitted by the patient or a medical worker. As a result, even if the pain situation is not recognized based on the endoscopy status information ESI, the pain status can be recognized because the input pain information INPN transmitted by the patient or a medical worker is prioritized, thereby outputting the pain situation information PSI. For example, if there is a pain situation due to individual differences in the patient or a pain situation unique to the patient, the pain situation may not be recognized based on the endoscopy status information ESI; however, in the present embodiment, since the input pain information INPN is prioritized, those pain situations can be recognized.

The endoscopy status information acquisition section 110 classifies the endoscope insertion status based on at least one of the endoscopic image, the insertion section shape information, and the operation recognition information, and outputs the endoscopy status information ESI including the classification results. The pain situation recognition section 130 recognizes whether or not the insertion status indicated by the classification result corresponds to a situation where a pain is caused, thereby recognizing the pain situation.

An endoscope insertion status is a predetermined insertion status that appears in the procedure to proceed with the endoscopy, and is identified, for example, by a predetermined position of the insertion section, a change in the predetermined position of the insertion section, a predetermined shape of the insertion section, a change in the predetermined shape of the insertion section, a predetermined operation of the endoscope, or any combination of two or more of them. For example, in colonoscopy, there are insertion methods such as the loop method, the shaft retention and shortening method, and the like, and, in each insertion method, there are insertion statuses, such as the shape of the insertion section or the operation, that appear when proceeding with the insertion procedure. In each of those insertion statuses, the next operation to be performed is fixed, and a pain is more likely to be caused in some specific insertion statuses from among those insertion statuses.

The endoscopy status information acquisition section 110 determines which of a plurality of insertion statuses corresponds to the insertion status based on at least one of the endoscopic image, the insertion section shape information, and the operation recognition information. The plurality of insertion statuses include, for example, a plurality of insertion statuses in which insertion support information should be presented and a plurality of insertion statuses in which a pain is caused. The pain situation recognition section 130 recognizes a situation where the patient is suffering a pain when the determined insertion status corresponds to an insertion status in which pain is caused. The insertion support information generation section 150 generates insertion support information indicating the next operation to be performed in the determined insertion status. At that time, the insertion support information generation section 150 changes the insertion support information according to whether or not a pain situation has been recognized.

According to the present embodiment, the insertion status of the endoscope is classified, thus enabling the pain situation recognition section 130 to recognize whether or not the insertion status corresponds to an insertion status where a pain is caused, thereby recognizing the pain situation. In addition, as described above, since certain insertion statuses that tend to cause pain in endoscopy are determined in advance, by using those insertion statuses as possible factors for classification, it is possible to determine whether or not the insertion status is an insertion status where a pain is caused.

The insertion support information generation section 150 generates insertion support information corresponding to the insertion status of the endoscope based on the endoscopy status information ESI. At this time, the insertion support information generation section 150 generates the first insertion support information as the insertion support information when the pain situation information PSI that does not indicate a situation where the patient is suffering a pain is input, and generates the second insertion support information as the insertion support information when the pain situation information PSI indicating a situation where the patient is suffering a pain is input. The second insertion support information is different from the first insertion support information.

Specifically, the operation presented by the second insertion support information is different from that presented by the first insertion support information. The first insertion support information indicates an operation for a situation with no pain, i.e., a normal operation in the insertion procedures. The second insertion support information indicates an operation for a situation where the patient is suffering a pain, i.e., a pain relief or pain avoidance operation that is different from the normal operation procedure described above. It is also possible to generate different kinds of insertion support information according to the pain level, in addition to according to the presence or absence of the pain situation. That is, the insertion support information generation section 150 may generate the second insertion support information when the pain situation recognition section 130 recognizes a pain situation with a first pain level, and may generate third insertion support information when the pain situation recognition section 130 recognizes a pain situation with a second pain level. The second pain level is higher or lower than the first pain level, and the third insertion support information is different from the first insertion support information or the second insertion support information.

According to the present embodiment, different kinds of insertion support information can be generated depending on whether or not the situation is a situation where the patient is suffering a pain. In other words, it is possible to present appropriate insertion support information in each of the situation where no pain is caused and the situation where a pain is caused.

The operation recognition information described above may include insertion section shape displacement information, which is information about the shape displacement of the endoscope insertion section. In this case, the endoscopy status information acquisition section 110 performs the above classification based on the insertion section shape information and insertion section shape displacement information. The shape displacement is the displacement in shape before and after the change in the shape of the endoscope insertion section. The shape displacement information may include either or both of the information about the direction of displacement and the information about the magnitude of displacement.

The shape of the endoscope insertion section changes as the endoscope is operated, and the patient may feel pain in response to the shape displacement at this time. According to the present embodiment, the classification is performed based on the insertion section shape displacement information; therefore, the pain situation caused by the shape displacement of the endoscope insertion section can be recognized.

As described above, the insertion section shape displacement information may include shape displacement amount information, which is information about the magnitude of the shape displacement. The endoscopy status information acquisition section 110 performs the above classification based on the insertion section shape information and the shape displacement amount information.

In certain insertion statuses, pain may be caused when the magnitude of shape displacement exceeds a predetermined value. According to the present embodiment, since the classification is performed based on the shape displacement amount information, the pain situation that is caused when the magnitude of the shape displacement exceeds a predetermined value can be recognized.

The input pain information INPN described above is at least one of patient facial expression information and pain communication information. The patient facial expression information is information about the patient's facial expression during endoscopy. Specifically, the camera captures the patient's facial expression, and the facial expression recognition section recognizes whether or not the patient's facial expression in the image indicates the presence of pain and outputs the result as the pain communication information. The pain communication information is information from a communication device operated by the patient or a medical worker. Specifically, the communication device is a device, such as a switch, a touch panel, or the like, which can be operated by the patient or a medical worker according to the pain situation of the patient. Each of the camera, the facial expression recognition section, and the communication device may be included in the insertion support system 100, or may be provided outside the insertion support system 100.

According to the present embodiment, the input pain information INPN can be acquired based on the patient's facial expression, the communication from the patient, the communication from a medical worker, or any combination of two or more of them. Therefore, as described above, it is possible to recognize the pain situation information PSI and generate insertion support information based on the input pain information INPN.

The pain situation described above is not limited to the presence/absence of pain. Specifically, the pain situation recognition section 130 may recognize the pain situation by distinguishing respective situations with different pain levels, different pain frequencies, or different degrees of pain avoidability from each other.

The pain level is the level of pain felt by the patient in the pain situation when a situation where the patient is suffering a pain is recognized. For example, the pain level varies according to the displacement amount of the endoscope insertion section in a certain insertion status. In this case, the pain level is determined based on the displacement amount. Alternatively, when the pain level varies according to the insertion status, each insertion status is associated with a pain level. When a certain insertion status is detected, the pain level associated with the detected insertion status is output.

The pain frequency is the frequency of recognition of the situation in which the patient is suffering a pain; for example, the number of times the pain situation is recognized per unit of time. This frequency may be either the frequency in the case where the same insertion status is repeated or frequency in the case where a plurality of insertion statuses are mixed. For example, the insertion support system 100 includes a memory (not shown), and the pain situation recognition section 130 stores the pain situation information PSI in the memory and detects the pain frequency by referring to the pain situation information PSI stored in the memory.

The pain avoidability is determined depending on the availability of pain-relieving or pain-avoiding operations in each pain situation. For example, if no pain-relieving or pain-avoiding operations are available in a given insertion status, the degree of pain avoidability is low. Further, if pain-avoiding operations are available in a given insertion status, the degree of pain avoidability is high. Furthermore, if a pain-relieving operation is available but no pain-avoiding operation is available in a given insertion status, the degree of pain avoidability is moderate. For example, when the pain situation recognition section 130 recognizes a certain pain situation, referring to the insertion support information associated with that pain situation allows the pain situation recognition section 130 to know selectable operations in the pain situation.

FIG. 2 is a second configuration example of an insertion support system 100. In FIG. 2, the insertion support system 100 further includes an examination condition information acquisition section 160. The same components as those already described will be omitted from the description as appropriate.

The examination condition information acquisition section 160 acquires examination condition information including at least one of endoscope kind information, patient information, and past examination information. The endoscope kind information is information about the kind of the endoscope insertion section used for endoscopy. The patient information is information about patient attributes. The past examination information is information about past endoscopies. The insertion support information generation section 150 generates the insertion support information based on the examination condition information. Further, the pain situation recognition section 130 also recognizes the pain situation based on the examination condition information.

Depending on the kind of the endoscope, the endoscope insertion section has different physical characteristics, such as thickness, hardness, and the like. For example, an endoscope usually stores an ID indicating its model, and the endoscope kind information can be acquired by acquiring the ID. Depending on the physical characteristics of the endoscope insertion section, such as its thickness, hardness, or the like, the likelihood of occurrence of pain situation, the pain level, or the like, varies. By acquiring the endoscope kind information, the pain situation recognition section 130 appropriately recognizes the pain situation according to the physical characteristics, such as the thickness, hardness, and the like, of the endoscope insertion section.

The patient information is, for example, the patient's gender, physique, body type, medical history, and the like. For example, the patient information can be acquired from information stored in electronic health records, as described later. It is also possible to input the patient information into the insertion support system 100 by a medical worker. The likelihood of occurrence of pain situation, the pain level, and the like, vary depending on the patient's gender, physique, body type, medical history, and the like. By acquiring the patient information, the pain situation recognition section 130 appropriately recognizes the pain situation according to the patient's gender, physique, body type, medical history, and the like.

The past examination information is examination information in the past in relation to the same site as that subjected to the current examination, for example, either or both of pain situations previously occurred, the insertion support information presented at that time. The past examination information may be examination information acquired in the past from the same patient as the patient being tested this time, or may be examination information acquired in the past from a plurality of patients. For example, the pain situation information PSI output by the pain situation recognition section 130 and the insertion support information generated by the insertion support information generation section 150 can be recorded as a log in a memory (not shown), and the past examination information can be acquired by referring to the log recorded in the past.

The processing of the insertion support system 100 described above may be implemented as an insertion support method as follows. The entity implementing the insertion support method is not limited to the insertion support system 100, but may be various systems or devices, such as the endoscope system described later. The insertion support method includes: acquiring the endoscopy status information ESI; receiving input of information of at least one of the input pain information INPN and the endoscopy status information ESI; acquiring the pain situation information PSI upon recognition of a pain situation; and generating insertion support information according to the pain situation information PSI and the endoscopy status information ESI.

In addition, some or all of the processes of the insertion support system 100 described above may be implemented by a program. In this case, the insertion support system 100 may be configured as follows.

The insertion support system 100 includes a memory for storing information and a processor that operates based on the information stored in the memory. The information includes, for example, a program and various types of data. The program describes some or all of the functions of the endoscopy status information acquisition section 110, the pain situation recognition section 130, the insertion support information generation section 150, and the examination condition information acquisition section 160. The processor executes the program to implement some or all of the functions of the endoscopy status information acquisition section 110, the pain situation recognition section 130, the insertion support information generation section 150, and the examination condition information acquisition section 160.

The processor includes hardware, and the hardware may include at least one of a circuit that processes a digital signal and a circuit that processes an analog signal. For example, the processor may include one or a plurality of circuit devices or one or a plurality of circuit elements mounted on a circuit board. The one or a plurality of circuit devices is, for example, an integrated circuit (IC). The one or a plurality of circuit elements is, for example, a resistor or a capacitor. For example, the processor may be a central processing unit (CPU). However, the processor is not limited to the CPU, and various processors such as a Graphics Processing Unit (GPU), a Digital Signal Processor (DSP), and the like, may be used. The processor may also be an integrated circuit device, such as an Application Specific Integrated Circuit (ASIC), a Field Programmable Gate Array (FPGA), or the like. The processor may include an amplifier circuit, a filter circuit, or the like that processes an analog signal. The memory may be a semiconductor memory such as an SRAM, a DRAM or the like, or may be a register. The memory may also be a magnetic storage device, such as a hard disk device or an optical storage device such as an optical disc device. For example, the memory stores therein computer-readable commands, and the processes (functions) of the sections of the insertion support system 100 are implemented with the processor executing the commands. These commands may be a command set included in a program, or may be commands to give operating instructions to the hardware circuit of the processor.

The program described above may be stored in, for example, a non-transitory information storage medium, which is a computer-readable medium. The information storage medium can be implemented by, for example, an optical disc, a memory card, an HDD, a semiconductor memory, or the like. The semiconductor memory is, for example, a ROM or a nonvolatile memory.

### 2. Endoscope System

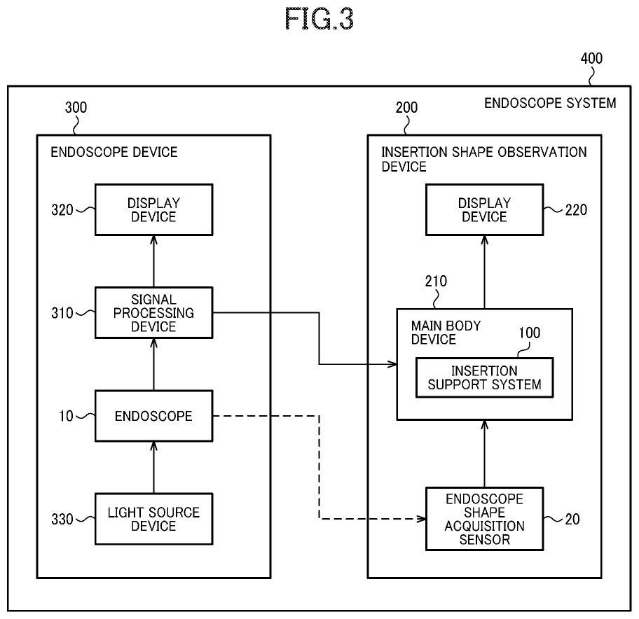

A detailed configuration example is described below. FIG. 3 is a configuration example of an endoscope system 400 including an insertion support system 100. The endoscope system 400 includes an endoscope device 300 and an insertion shape observation device 200.

The endoscope device 300 includes an endoscope 10, a light source device 330, a signal processing device 310, and a display device 320. The endoscope 10, which is also called a scope, is inserted into the patient's body to capture images inside the patient's body. The light source device 330 generates and controls the illumination light, which is guided by a light guide to the distal end of the endoscope 10, and emitted from the distal end of the endoscope 10. The signal processing device 310 generates endoscopic images by processing the image signals output by the endoscope 10. The signal processing device 310 also obtains the ID or the like of the endoscope 10, as the endoscope kind information. The display device 320 displays endoscopic images generated by the signal processing device 310.

The insertion shape observation device 200 includes the endoscope shape acquisition sensor 20, the main body device 210, and the display device 220. The endoscope shape acquisition sensor 20 detects the magnetic field of the source coil provided in the endoscope insertion section. The main body device 210 acquires the position and the shape of the endoscope insertion section based on the detection signal from the endoscope shape acquisition sensor 20, and outputs the images showing the position and the shape of the endoscope insertion section to the display device 220. The display device 220 displays the images output by the main body device 210. The display device 220 and the display device 320 are also called a monitor and are liquid crystal displays or the like. A single display device may be provided in the endoscope system 400, and the endoscope device 300 and the insertion shape observation device 200 may share the single display device.

The insertion support system 100 is provided in the main body device 210. The endoscopic image and the endoscope kind information from the signal processing device 310 and the information about the position, the shape, and the like of the endoscope insertion section acquired by the main body device 210 are input to the insertion support system 100. The insertion support system 100 may be located anywhere within the endoscope system 400.

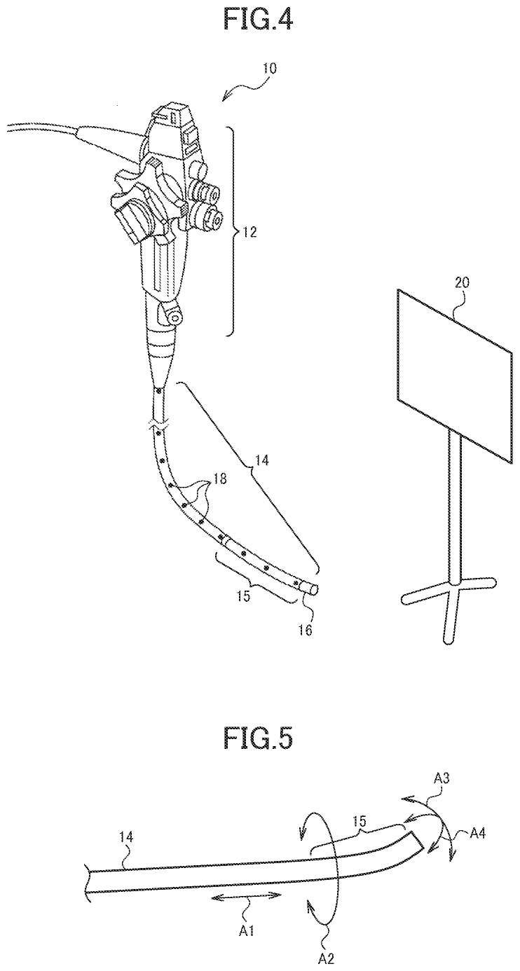

FIG. 4 is a configuration example of the endoscope 10 and the endoscope shape acquisition sensor 20. As shown in FIG. 4, the endoscope 10 includes an operation section 12, an endoscope insertion section 14, and a source coil 18.

The endoscope insertion section 14 has a flexible, elongated shape and includes a rigid section 16 at its distal end and an angle-operable bending section 15. The rigid section 16 is equipped with an imaging device, an illumination lens, a water supply port, an air supply port, a forceps port and the like.

The operation section 12 is a device for allowing the user to operate the endoscope 10, and includes, for example, a grip section, an angle operating dial, an air/water supply button, and the like. FIG. 5 shows an explanatory view of operation of the endoscope. As shown in A1, the user pushes the grip section in the longitudinal direction of the endoscope insertion section 14, so that the endoscope insertion section 14 is inserted. This action is called a push operation. When the user pulls the grip section in the longitudinal direction of the endoscope insertion section 14, the endoscope insertion section 14 is pulled out. This action is called a pull operation. As shown in A2, the user turns the grip section in the circumferential direction of the endoscope insertion section 14, so that the endoscope insertion section 14 rotates in the circumferential direction. This action is called a torque operation. When viewed from the grip section in the direction of the endoscope insertion section 14, a clockwise torque operation is called a right torque operation and a counterclockwise torque operation is called a left torque operation. As shown in A3 and A4, when the user operates the angle operating dial, the bending section 15 of the endoscope insertion section 14 bends up, down, left, and right. This action is called an angle operation. The vertical (up and down) angle operation and the horizontal (left and right) angle operation can be operated independently.

The source coil 18 generates a magnetic field. For example, a plurality of source coils 18 are provided in the endoscope insertion section 14 at predetermined intervals. The endoscope shape acquisition sensor 20 detects the magnetic field from each source coil 18, and the main body device 210 of the insertion shape observation device 200 detects the position of each source coil 18 based on the detection signal, thereby detecting the position of each section of the endoscope insertion section 14. The main body device 210 also detects the shape of the endoscope insertion section 14 based on the detected positions of the plurality of source coils 18. The sensing method in the observation of the insertion shape is not limited to the method using magnetic fields, but may also be a method using electromagnetic waves, ultrasonic waves, lights, and the like, for example.

### 3. First Detailed Configuration Example of Insertion Support System

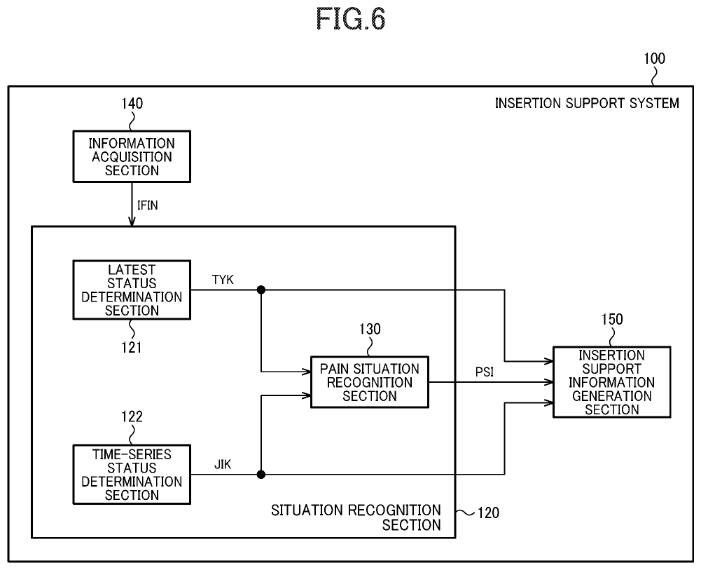

FIG. 6 is a first detailed configuration example of the insertion support system 100. The insertion support system 100 includes an information acquisition section 140, a situation recognition section 120, and the insertion support information generation section 150. In this configuration example, for example, a part of the information acquisition section 140 and the latest status determination section 121 correspond to the endoscopy status information acquisition section 110 in FIGS. 1 and 2, and a part of the information acquisition section 140 and the time-series status determination section 122 correspond to the examination condition information acquisition section 160 in FIG. 2. The same components as those already described will be omitted from the description as appropriate.

The information acquisition section 140 acquires various types of information IFIN used to recognize a pain situation. The information IFIN is, for example, an endoscopic image, insertion section shape information, input pain information, endoscope kind information, patient information, past examination information, or any combination of two or more of them.

The situation recognition section 120 recognizes the latest status, the time-series status and the pain situation from the information IFIN and outputs the latest status information TYK, the time-series status information JIK and the pain situation information PSI. The situation recognition section 120 includes the latest status determination section 121, the time-series status determination section 122, and the pain situation recognition section 130.

The latest status determination section 121 recognizes the current endoscope insertion status from endoscopic images or insertion section shape information or other information, and outputs the result as the latest status information TYK. The “current” means not only the moment right now, but also the most recent time including the current time. For example, the most recent time is a time period long enough to recognize a series of movements of the insertion section when the operation or operation procedure currently performed is identified by the series of movements of the insertion section.

The time-series status determination section 122 recognizes the insertion status including the recognition results of status in the past in a time-series manner from the past examination information or other information, and outputs the result as the time-series status information JIK. The “past” means the time before the “most recent time” described above, and may be either the time during the current endoscopy or time at or before the previous endoscopy.

The pain situation recognition section 130 recognizes a situation where pain is likely to be caused or a situation where pain has been caused from the latest status information TYK and the time-series status information JIK, and outputs the result as the pain situation information PSI. By using not only the latest status information TYK but also the time-series status information JIK, the pain situation recognition section 130 also determines the current pain situation based on past recognition results.

Based on the latest status information TYK, the time-series status information JIK, and the pain situation information PSI, the insertion support information generation section 150 generates insertion support information suitable for the situation indicated by these information items. Such a presentation of a guide suitable for the situation provides a support in inserting the endoscope with less pain.

As an example, the situation recognition section 120 is implemented by machine learning using a neural network or the like. More specifically, the memory (not shown) stores a program that describes an inference algorithm and parameters used for the inference algorithm, as the information of the trained model. Then, the processor performs the processing based on the information of the trained model. That is, the processor executes the program using the parameters stored in the memory, thereby executing the processing of the situation recognition section 120. The entire situation recognition section 120 may be implemented by a single trained model, or each of the latest status determination section 121, the time-series status determination section 122, and the pain situation recognition section 130 may be implemented by individual trained models. Further, only a part of the latest status determination section 121, the time-series status determination section 122, and the pain situation recognition section 130 may be implemented by trained models.

For example, a neural network may be used as the inference algorithm. The weight coefficients of the inter-node connections in the neural network correspond to the parameters. The neural network includes an input layer to which input data is entered, an intermediate layer for performing a calculation process with respect to the data entered via the input layer, and an output layer for outputting a recognition result based on the calculation result output from the intermediate layer. The inference algorithm is not limited to a neural network, and various types of machine learning techniques for use in recognition process may be used. In the following, a training process is described by referring to an exemplary case where the entire situation recognition section 120 is implemented by a single trained model. In this case, the input data is the information IFIN, and the recognition results are the latest status information TYK, the time-series status information JIK, and the pain situation information PSI. The training device that executes the learning process is, for example, an information processing device such as a PC. The training device generates a trained model by inputting training data into a training model, and providing feedback to the training model based on the recognition result. The training data contains multiple sets of data, each set containing input data and correct answer data. The correct answer data is the recognition result that is supposed to be obtained in response to the input data. The correct answer data is prepared in advance, for example, by a medical worker.

In the following, an example where the insertion support system 100 is applied to a colonoscope is described; however, the application of the insertion support system 100 is not limited to colonoscopes.

Examples (a) to (c) of the latest status information TYK and example (d) of the time-series status information JIK are shown below.

(a) The latest status determination section 121 performs an image recognition process with respect to an endoscopic image to acquire the following information.

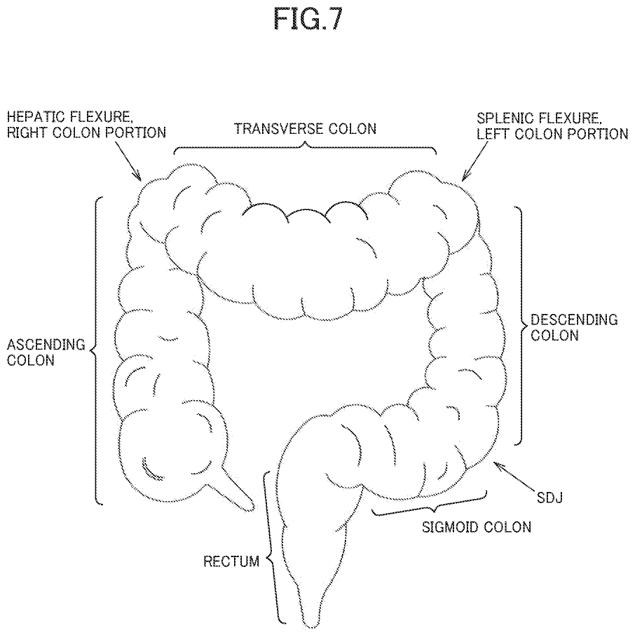

a1: The site where the endoscope insertion section is present. FIG. 7 is an explanatory view of a large intestine site. The large intestine site includes, from the cecum to the anus, the ascending colon, hepatic flexure, transverse colon, splenic flexure, descending colon, SDJ, sigmoid colon, and rectum. The area near the hepatic flexure is also called a right colon portion, and the area near the splenic flexure is also called a left colon portion. SDJ stands for Sigmoid Descending colon Junction, which is the boundary between the sigmoid colon and the descending colon. The site is recognized from the image by using the different features of the image depending on the site. The length of the portion of the endoscope insertion section that is inserted into the patient can be acquired from the insertion section shape information, and the site may also be assumed by this insertion length.

a2: Optimal distance, earthen tubular shape, red ball, or residue. The optimal distance and earthen tubular shape refer to the distance between the distal end of the insertion section and the intestinal wall in the shaft retention and shortening method. The optimal distance refers to the optimal distance between the insertion section and the intestinal wall with which the folds can be flipped by either or both of the angle operation and the torque operation. The optimal distance can be created by aspiration, push or pull operations. With such an optimal distance, the folds can be crossed without a push operation, thereby reducing the pain caused by the push operation. The earthen tubular shape refers to a condition where the distance is not optimal, i.e., the distal end of the insertion section is distant from the intestinal wall. The red ball refers to a state in which the distal end of the insertion section is in contact with the intestinal wall mucosa; in this state, because of the contact, the image appears reddish due to the red color of the mucosa. The residue refers to a state where water or the like remains in the intestinal wall.

a3: Intestinal movement. The intestinal movements include advancing, parallel, rotating, and peristaltic movements. The advancing/receding movement is the relative movement of the endoscope insertion section and the intestinal tract in the optical axis direction. The parallel movement is the relative movement of the endoscope insertion section and the intestinal tract in a direction perpendicular to the optical axis direction. The rotating movement is the relative rotation of the endoscope insertion section and the intestinal tract around the optical axis direction as the central axis. The peristaltic movement is a movement of the large intestine to translocate its contents.

a4: Suction or air supply. The suction refers to suction of the gas in the large intestine. By the suction, the intestinal wall is drawn to the distal end of the insertion section. The air supply is sending of gas into the large intestine. The air supply causes the large intestine to expand and the intestinal wall to separate from the distal end of the insertion section.

(b) The latest status determination section 121 performs a recognition process with respect to the insertion section shape information to acquire the following information.

b1: Shape of endoscope insertion section. The “shape” herein means a shape at a given moment, such as the present.

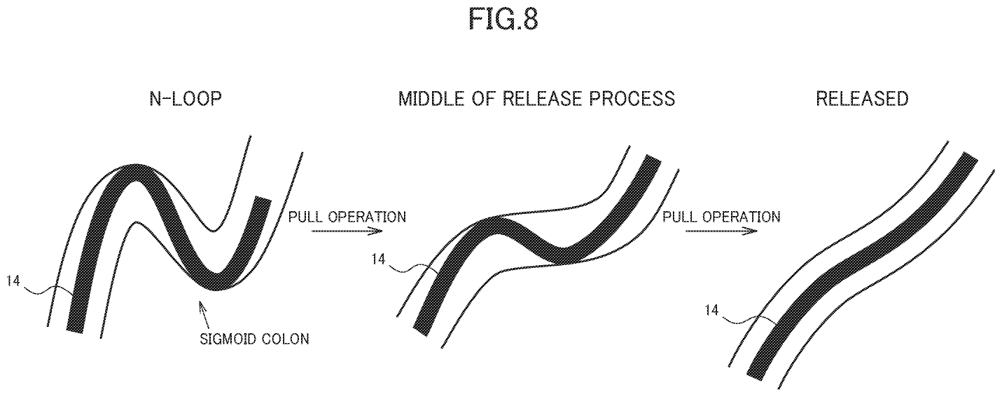

b2: Type of loop in loop method. It also means the shape transition of the endoscope insertion section during, before, and after the loop method. The types of loop include N-loop, a-loop, reverse a-loop, and y-loop, which are identified by the loop shape formed by the endoscope insertion section. FIG. 8 is an example of shape transition of the endoscope insertion section. Herein, the shape transition upon the release of N-loop is described as an example. The loop method is used to pass the endoscope insertion section through the sigmoid colon. When the N-loop is formed, the endoscope insertion section has substantially an N-shape. The N-loop is gradually released as the pull operation is performed. When the N-loop is released, the endoscope insertion section becomes substantially a straight line. In the middle of the release process, the endoscope insertion section has a shape between the substantially N-shape and the substantially straight line. By recognizing this shape transition, it is possible to recognize that the N-loop has been properly released.

b3: Shape transition of endoscope insertion section during, before, and after the shaft retention and shortening method. The shaft retention and shortening method is used to pass the endoscope insertion section through the sigmoid colon. In the shaft retention and shortening method, an angle operation is performed to cross the folds of the intestinal wall, and a torque operation is performed to fold up the folds. These operations are repeated to pass the endoscope insertion section through the sigmoid colon. The shape transition caused by these operations are detected.

b4: Shape transition of endoscope insertion section during transverse colon operation. The transverse colon operation is a shortening of mid-trans of transverse colon. As in the loop method or the like, the shape transition caused by the transverse colon operation is detected.

b5: Deflection of the endoscope insertion section or extension of intestinal wall by the endoscope insertion section. The deflection of the endoscope insertion section refers to deflection of the middle of the insertion section with its distal end staying still when a push operation is performed. The extension of the intestinal wall by the endoscope insertion section refers to pushing of the free colon of the large intestine by the endoscope insertion section, causing the intestinal wall between the pushed portion and the fixed colon of the large intestine to extend. In FIG. 7, the free colons are the sigmoid colon and the transverse colon, and the fixed colons are the rectum, the ascending colon, and the descending colon.

(c) The latest status determination section 121 acquires the following operation recognition information based on the shape displacement of the endoscope insertion section.

c1: Whether or not to perform a pushing, pulling, torque, or angle operation during shaft retention and shortening method, loop method, or shortening. Based on either or both of the direction of the displacement and the displacement amount of the endoscope insertion section, it is determined whether or not a pain is caused when the operation is performed or continued.

(d) The time-series status determination section 122 acquires the following information based on the past examination information.

d1: Insertion support information, image recognition results, shape recognition results, or operation recognition information generated in the past. These items of information are acquired by retrieving the past examination information accumulated in the storage device.

The following describes pain situations recognized based on the latest status information TYK and the time-series status information JIK described above.

The pain situation recognition section 130 recognizes, based on the endoscopy status information ESI, a situation where a pain is caused by the extension of the intestinal wall of the large intestine, a situation where a pain is caused by the mesentery being pulled, a situation where a pain is caused by the endoscope insertion section pushing the intestinal wall, or a situation where a pain is caused due to the relationship between the fixed colon of the large intestine and the endoscope insertion section, thereby recognizing the pain situation. The “relationship between the fixed colon of the large intestine and the endoscope insertion section” is the relationship in which the endoscope insertion section pushes or pulls the fixed colon by the operation of the endoscope. This relationship is estimated not from the force but from the position, shape, displacement of the position, displacement of the shape, or the like of the endoscope insertion section.

The above four situations are the main insertion statuses causing pain in the patient during colonoscopy. By enabling the pain situation recognition section 130 to recognize these insertion statuses, it is possible to recognize a pain situation where the patient is suffering a pain.

Specific examples of pain situations (1) to (23) are shown below.

(1) A situation where the push operation is performed when a loop is not formed. This pain situation is an insertion status in which the intestinal wall is extended by the push operation when no loop is formed.

(2) A situation where a right torque operation is performed when a reverse a-loop is formed. The reverse a-loop is released by a left torque operation; however, conversely, if a right torque operation is performed, the loop is not released and pain may be caused. This pain situation is the insertion status in which the reverse a-loop is not released because of the right torque operation.

(3) A situation where the push operation is performed when a loop is formed. This pain situation is an insertion status in which the intestinal wall is extended by the push operation when an N-loop is formed.

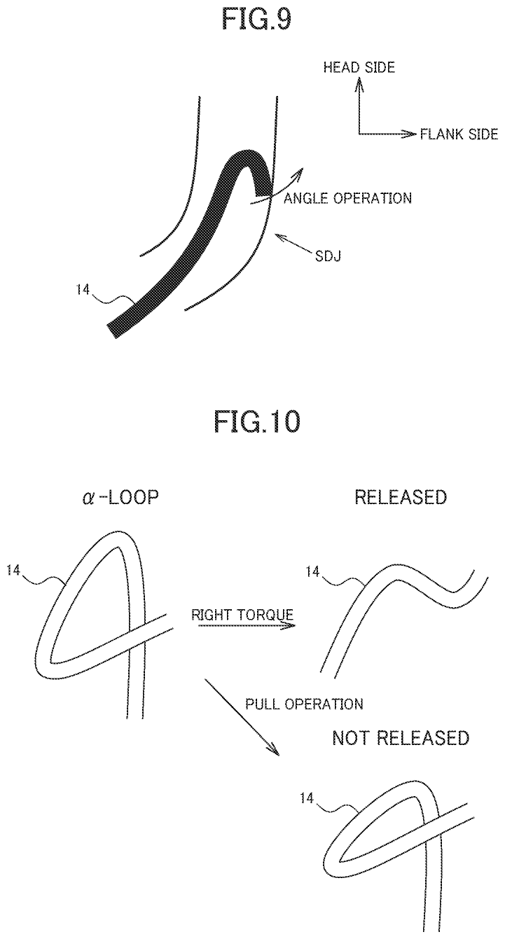

(4) A situation where a push up operation to the flank or head side is performed near the SDJ by an angle operation of the endoscope. FIG. 9 shows an explanatory view of this pain situation. Since the SDJ is near the boundary between the sigmoid colon and the descending colon, when a push up operation is performed near the SDJ, the descending colon, which is a fixed colon, is pushed, and this may cause pain. The position of the source coil 18 provided in the endoscope insertion section 14 is detected using the position of the endoscope shape acquisition sensor 20 as a reference. Therefore, based on the relative positional relationship between the patient and the endoscope shape acquisition sensor 20, the flank and head directions in the detected insertion section shape can be determined.

(5) A situation where a push up operation to the head side is performed near the SDJ by a torque operation and an angle operation of the endoscope. As in (4), pushing the descending colon, which is a fixed colon, may cause pain.

(6) A situation where an operation is performed near the SDJ with the endoscope insertion section being bent. This pain situation is an insertion status in which the fixed colon is pushed because the operation is performed near the SDJ, which is near the fixed colon. This is, for example, a situation where an operation is performed with an angle when, for example, an N-loop is formed.

(7) A situation where a pull operation is performed when the endoscope forms a loop. FIG. 10 shows an explanatory view of this pain situation. Although a-loop is described herein as an example, the same applies to other loops. An a-loop is released by a right torque operation. When a pull operation is performed during the formation of an a-loop, the loop is not released and pain may be caused.

(8) A situation where a pull operation is performed when a loop is not formed. This pain situation is an insertion status in which, when a loop is not formed, the pull operation is performed to an excessive extent or in the wrong direction.

(9) A situation where the operation is performed in the presence of adhesion. This pain situation is an insertion status in which a part of the endoscope insertion section is caught in the intestinal wall during the pull operation and thus does not move. In the portion of the intestinal wall in which a part of the endoscope insertion section is caught, there may be adhesion. For example, the distal end of the endoscope insertion section is caught in the adhesion portion. In this case, the distal end does not move by the pull operation, and the shapes of other parts change. It is also possible that the endoscopic image does not change. By recognizing these situations, it is possible to recognize this pain situation.

(10) A situation where a push up operation is performed against the vicinity of the boundary between the fixed colon and the free colon by a push operation or pull operation. The boundary between the fixed colon and the free colon is the SDJ or the splenic flexure. As in (4), pushing a fixed colon may cause pain.

(11) A situation where a push operation on the splenic flexure is performed. Because the diaphragm is located at the head side of the splenic flexure, pressing the splenic flexure against the diaphragm by an push operation may cause pain.

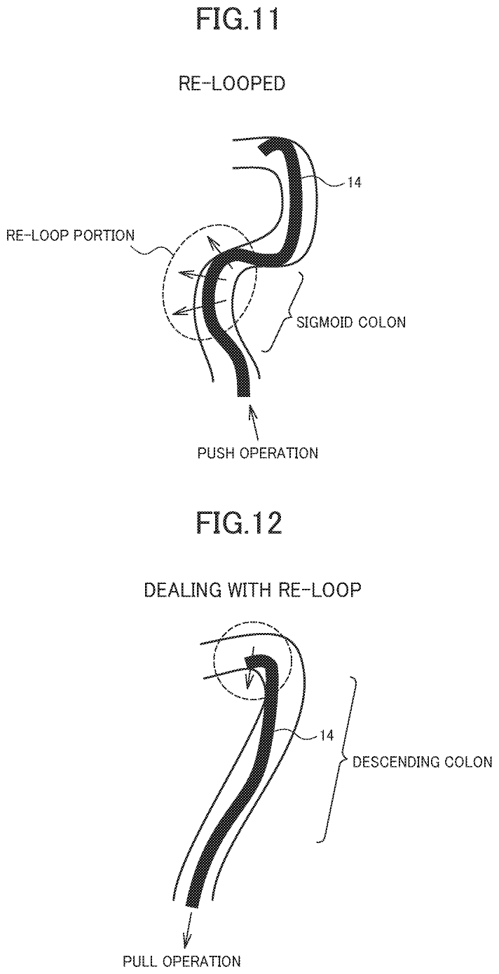

(12) A situation where a push operation is performed when the distal end of the endoscope insertion section is present in any of the splenic flexure, the transverse colon, and the hepatic flexure and a re-loop is formed in the sigmoid colon. FIG. 11 shows an explanatory view of this pain situation. Herein, a case where the distal end of the endoscope insertion section is present in the splenic flexure is shown. When a push operation is performed to pass the insertion section through the splenic flexure, the insertion section near the sigmoid colon may be deflected with its distal end caught in the splenic flexure. This portion with such a deflected insertion section near the sigmoid colon is called a re-loop. In this state, by performing a push operation to a certain extent or more, the re-loop expands, causing extension of the intestinal wall. This pain situation can be recognized by recognizing that the distal end of the endoscope insertion section has not moved and that the re-loop has been displaced to a certain extent or more.

(13) A situation where the distal end of the endoscope insertion section is present in any of the splenic flexure, the transverse colon, and the hepatic flexure and a pull operation is performed upon dealing with a re-loop in the sigmoid colon. FIG. 12 shows an explanatory view of this pain situation. Herein, a case where the distal end of the endoscope insertion section is present in the splenic flexure is shown. Although the re-loop is released by a pull operation, if the pull operation is performed with the bend at the distal end of the insertion section caught in the upper end of the descending colon, pain may be caused. As in (12), this pain situation can be recognized by the difference between the movement of the distal end of the insertion section and the movements of other portions.

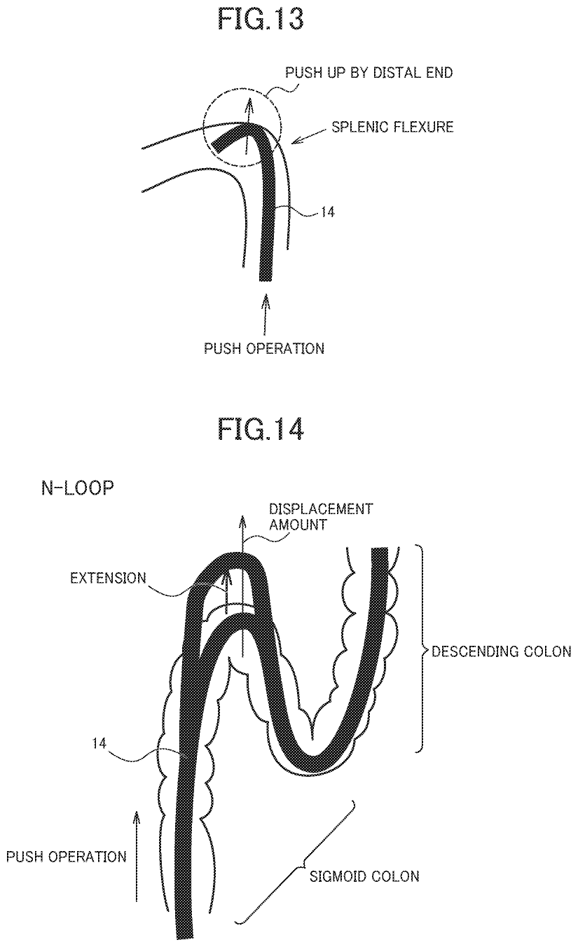

(14) A situation where the left colon portion is pushed up from the splenic flexure by a push operation of the endoscope. FIG. 13 shows an explanatory view of this pain situation. By performing a push operation with the endoscope insertion section in contact with the splenic flexure, the splenic flexure is pushed upward. This may cause extension of the left colon portion or push it against the diaphragm.

(15) A situation where a shortening operation has caused the descending colon to the splenic flexure to be pulled down to the anus side or caused the right colon portion to bounce up to the head side. When the pull operation is performed in the shortening of the mid-trans, the distal end of the insertion section in the right colon portion is raised to the head side and the insertion section near the splenic flexure is lowered to the anus side. As a result, the ascending colon, which is a fixed colon, is pulled, or the descending colon, which is a fixed colon, is pushed, and this may cause pain.

(16) A situation where the splenic flexure is pushed up to the head side by a push operation at the time of mid-trans insertion. When the endoscope insertion section is located at the position where the transverse colon is hung down, the push operation may cause the insertion section near the splenic flexure to be deflected to the head side, thereby pushing the splenic flexure upward.

(17) A situation where the right colon portion is pulled toward the left flank side by an angle operation during the shortening operation.

(18) A situation where the hepatic flexure is pushed up by an angle operation.

(19) A situation where the hepatic flexure is pushed up by a push operation.

(20) A situation where, when the distal end of the endoscope insertion section is in the hepatic flexure, the splenic flexure is pushed up by a push operation.

(21) After reaching the hepatic flexure, the splenic flexure is pulled to the anus side by a pull operation.

(22) A situation where the splenic flexure is pulled to the anus side by a pull operation in the splenic flexure.

(23) A situation where the intestinal tract inflates by air supply.

The pain situation recognition section 130 may recognize a pain situation when the displacement amount exceeds a predetermined value in each situation. As shown in FIG. 14, in the situation (3) above, a pain situation may be determined when an N-loop is detected and the displacement amount of the convex portion of the loop is equal to or greater than a predetermined value. Since the degree of extension changes with the displacement amount, the pain situation is recognized when the displacement amount becomes a value with which a certain degree of extension or more occurs.

The pain situation recognition section 130 may also determine a pain level based on the displacement amount in each situation. For example, in FIG. 14, the pain level may be determined as a first pain level when the displacement amount of the convex portion of the loop is equal to or greater than a first predetermined value, and the pain level may be determined as a second pain level when the displacement amount of the convex portion of the loop is equal to a second predetermined value. When the second predetermined value is greater than the first predetermined value, the second pain level indicates greater pain than that of the first pain level.

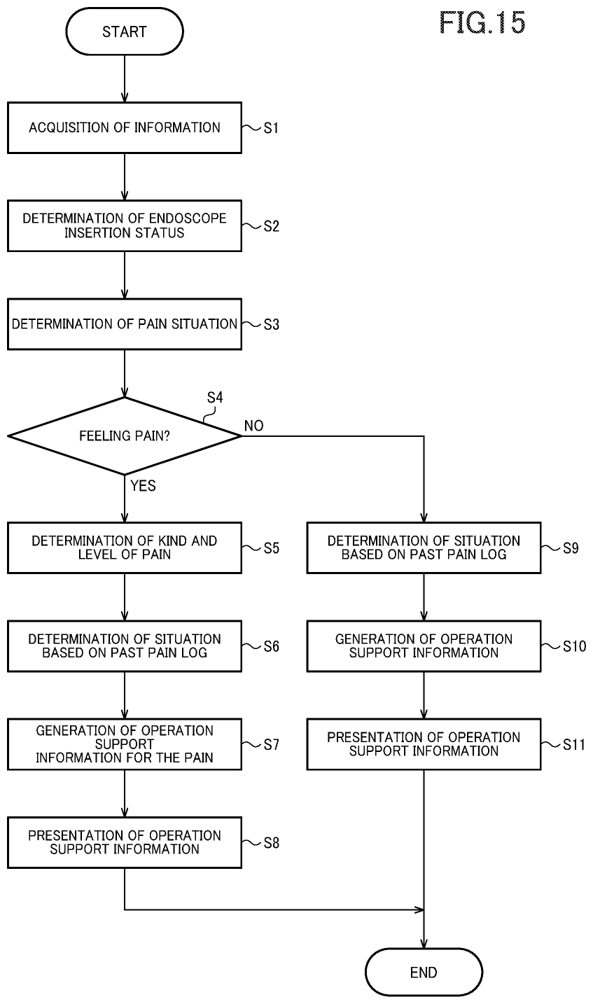

FIG. 15 is a flowchart of the processing performed by the insertion support system 100 in the first detailed configuration example. In this flowchart, the insertion support information generated in the step S7 corresponds to the first insertion support information generated in the situation where the patient is not suffering a pain described in FIG. 1. The insertion support information generated in the step S8 corresponds to the second insertion support information that is generated in the case of a pain situation described in FIG. 1.

In the step S1, the information acquisition section 140 acquires the information IFIN. In the step S2, the latest status determination section 121 and the time-series status determination section 122 determine the insertion status of the endoscope based on the information IFIN. In the steps S3 and S4, the pain situation recognition section 130 determines the presence or absence of a pain situation based on the latest status information TYK and the time-series status information JIK.

In the step S4, when the pain situation recognition section 130 determines the presence of a pain situation, in the step S5, the pain situation recognition section 130 determines the kind of pain and the pain level based on the insertion status determined in the step S2. The pain situation recognition section 130 also determines the frequency of pain and the time when pain occurred based on the insertion status determined in the step S2. In the step S6, pain situations occurred in the past are acquired based on the insertion status determined in the step S2. In the step S7, the insertion support information generation section 150 generates insertion support information based on the pain situation recognized by the pain situation recognition section 130 in the steps S5 and S6. The insertion support information herein refers to information indicating operations for reducing pain or operations for avoiding pain. In the step S8, the insertion support information generation section 150 presents the generated insertion support information by outputting it to a display device or the like.

The pain situations (1) to (23) described above correspond to the kind of pain. The operations for reducing pain or the operations for avoiding pain are, for example, operations opposite to those corresponding to the conditions in which pain has occurred or pain is likely to occur, or manual compression by an operation assistant, or repositioning of the body position of the patient, and the like. As an example, in (3) above, when the pain situation is caused by a push operation with a N-loop, a pull operation or manual compression is presented.

In the step S7, the insertion support information generation section 150 changes the content of operation to be presented according to the kind or level of the pain caused. The insertion support information generation section 150 changes the content of operation to be presented according to how many times the same pain occurred in the past, how long the same pain continued in the past, and whether or not different kinds of pain occurred in the past. The insertion support information generation section 150 may also suggest a change of scope, a change of surgeon, or discontinuation of the examination if the pain frequency is high. These items of pain information may also be recorded in electronic health records.

When the pain situation recognition section 130 determines absence of a pain situation in the step S4, pain situations occurred in the past are acquired in the step S9 based on the insertion status determined in the step S2. In the step S10, the insertion support information generation section 150 generates insertion support information based on the pain situation recognized by the pain situation recognition section 130 in the step S9. The insertion support information herein refers to information indicating normal operations, operations for avoiding easy occurrence of pain, or operations for avoiding occurrence of pain in advance. In the step S11, the insertion support information generation section 150 presents the generated insertion support information by outputting it to a display device or the like.

The operations for avoiding easy occurrence of pain or the operations for avoiding occurrence of pain in advance are, for example, methods that do not stretch the intestine, such as the shaft retention and shortening method.

In the step S9, if there were pains in the past in the same insertion status, even if there is no pain at present, the insertion support information generation section 150 changes the insertion support information to information of an operation that does not cause the same pain.

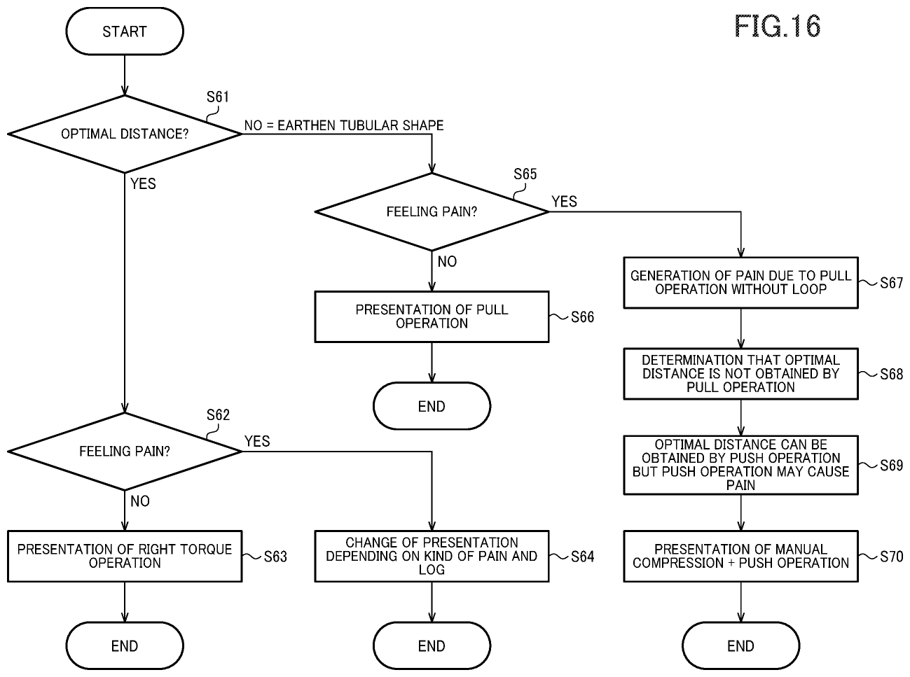

FIG. 16 is a flowchart showing a specific example of recognition of pain situation and generation of insertion support information according to the recognition results. In the following, the shaft retention and shortening method is described as an example; however, the process flow is set up for each insertion method or each of the pain situations (1) to (23) described above.

In the step S61, the latest status determination section 121 determines whether or not the distance is the optimal distance based on the information IFIN.

When the latest status determination section 121 determines in the step S61 that the distance is the optimal distance, in the step S62, the pain situation recognition section 130 determines whether or not a pain situation is caused based on the insertion status recognized by the latest status determination section 121 and the time-series status determination section 122. When the pain situation recognition section 130 determines in the step S62 that it is not a pain situation, in the step S63, the insertion support information generation section 150 generates insertion support information indicating a right torque operation. When the pain situation recognition section 130 determines in the step S62 that it is a pain situation, in the step S64, the insertion support information generation section 150 generates insertion support information having contents according to the kind of pain or pain situations in the past, or the like.

When the latest status determination section 121 determines in the step S61 that the distance is not the optimal distance, in the step S65, the pain situation recognition section 130 determines whether or not a pain situation is caused based on the insertion status recognized by the latest status determination section 121 and the time-series status determination section 122. When the pain situation recognition section 130 determines in the step S65 that it is not a pain situation, in the step S66, the insertion support information generation section 150 generates insertion support information indicating a pull operation. When the pain situation recognition section 130 determines in the steps S65 and S67 that there is a pain situation caused by a pull operation that is performed when a loop is not formed, in the steps S68 and S69, the pain situation recognition section 130 determines that the optimal distance cannot be obtained if a pull operation is performed, and that the optimal distance can be obtained if a push operation is performed but the push operation may cause pain. In the step S70, the insertion support information generation section 150 generates insertion support information indicating combined use of manual compression and a push operation.

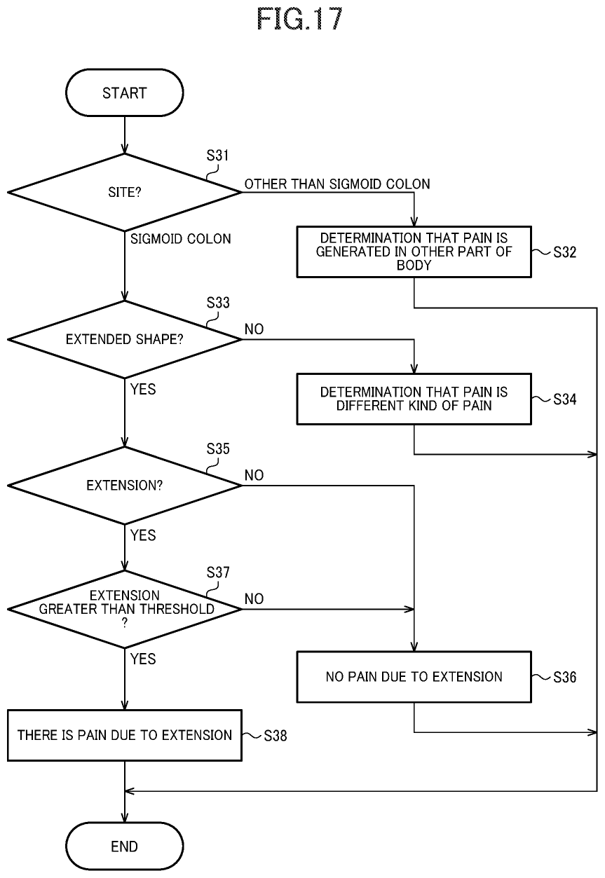

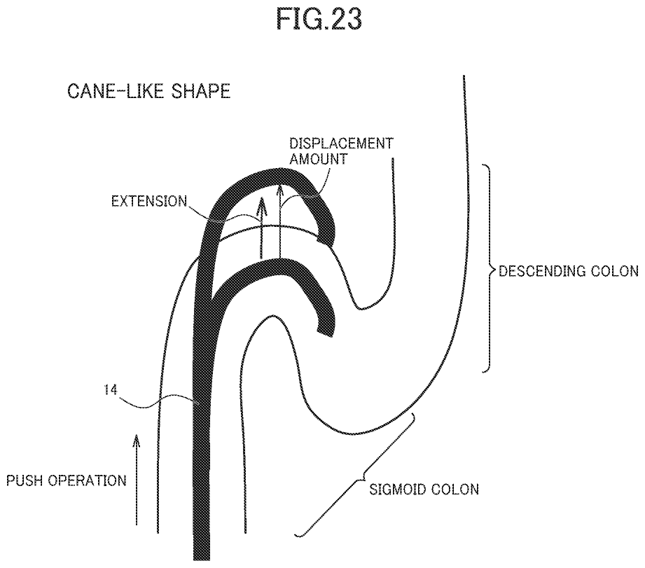

FIG. 17 is a flowchart showing a specific example of recognition of pain situation. In the following, as shown in FIG. 23, a pain situation where extension occurs in the sigmoid colon is described as an example.

In the step S31, the latest status determination section 121 determines the site where the endoscope insertion section is present based on an endoscopic image and insertion section shape information. Specifically, the latest status determination section 121 performs image recognition with respect to the endoscopic image and performs shape recognition with respect to the insertion section shape information, and determines the site where the endoscope insertion section is present based on the image recognition results and the shape recognition results.

If the latest status determination section 121 determines in the step S31 that the endoscope insertion section is present outside the sigmoid colon, in the step S32, the pain situation recognition section 130 determines that the pain situation occurs in another part of the body.

When the latest status determination section 121 determines in the step S31 that the endoscope insertion section is present in the sigmoid colon, in the step S33, the latest status determination section 121 determines whether or not the endoscope insertion section has an extended shape. The extended shape herein refers to a cane-like shape that is convex in the head side while being bent to the left flank side, as shown in FIG. 23.

When the latest status determination section 121 determines in the step S33 that the endoscope insertion section does not have an extended shape, in the step S34, the pain situation recognition section 130 determines that the pain situation is another kind of pain situation.

When the latest status determination section 121 determines in the step S33 that the endoscope insertion section has an extended shape, in the step S35, the latest status determination section 121 determines whether or not the extension has occurred. Specifically, the latest status determination section 121 determines the presence of extension based on either or both of the fact that the endoscope insertion section is being displaced to the head side while maintaining its extended shape and the fact that the endoscopic image is being stopped or went backward in the section where the extension was recognized from the latest recognition information.

When the latest status determination section 121 determines in the step S35 that no extension has occurred, in the step S36, the pain situation recognition section 130 determines that no pain situation due to extension has been generated.

When the latest status determination section 121 determines in the step S35 that extension has occurred, in the step S37, the pain situation recognition section 130 determines whether or not the extension is greater than the threshold value. Specifically, the pain situation recognition section 130 determines whether or not the displacement amount of the convex portion of the endoscope insertion section is greater than the threshold value, as shown in FIG. 23.

When the pain situation recognition section 130 determines in the step S37 that the extension is not greater than the threshold value, in the step S36, the pain situation recognition section 130 determines that no pain situation due to extension has been generated.

When the pain situation recognition section 130 determines in the step S37 that the extension is greater than the threshold value, in the step S38, the pain situation recognition section 130 determines that a pain situation due to extension has been generated.

### 4. Second Detailed Configuration Example of Insertion Support System

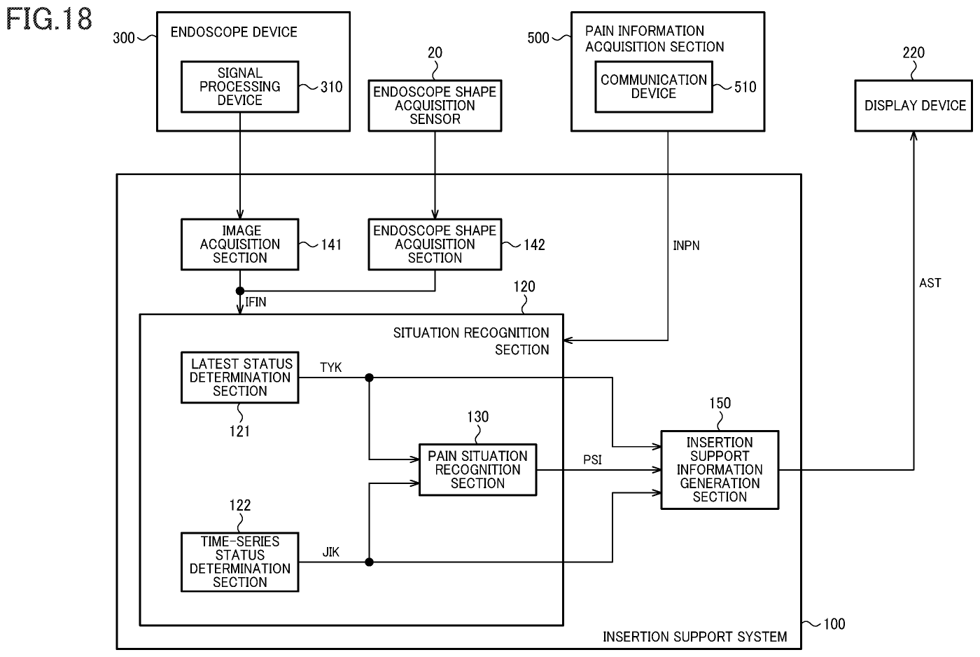

FIG. 18 is a second detailed configuration example of the insertion support system 100. The insertion support system 100 includes an image acquisition section 141, an endoscope shape acquisition section 142, the situation recognition section 120, and the insertion support information generation section 150. In this configuration example, for example, the image acquisition section 141, the endoscope shape acquisition section 142, and the latest status determination section 121 correspond to the endoscopy status information acquisition section 110 in FIGS. 1 and 2, and the time-series status determination section 122 corresponds to the examination condition information acquisition section 160 in FIG. 2. The same components as those already described will be omitted from the description as appropriate.

The pain information acquisition section 500 is included in the endoscope system 400. For example, when the insertion support system 100 is included in the insertion shape observation device 200, the pain information acquisition section 500 may be included in the insertion shape observation device 200.

The pain information acquisition section 500 includes a communication device 510 that can be operated by the patient or a medical worker according to the pain situation of the patient. The communication device 510 is, for example, a switch, a touch panel, or the like. In an exemplary case where the patient operates a switch, the patient is given a switch and asked to press the switch when he/she feels pain. The pain situation recognition section 130 recognizes the occurrence of pain based on the information from the switch, and the insertion support information generation section 150 presents insertion support information based on the recognition result. The switch may be a one-step switch, or a two-step switch that enables the patient to indicate the intensity of the pain. The switch may be one capable of communicating only the presence or absence of pain, or one capable of communicating information other than the presence or absence of pain, such as intensity or duration of the pain. It is also possible that the switch is held by a nurse or other assistants, enabling them to press the switch when the patient complains of pain.

Further, the pain information acquisition section 500 may also include a camera that captures an image of patient's facial expression, and pain may be recognized by recognizing that the captured image shows a painful facial expression. Further, the pain information acquisition section 500 may also include a microphone that acquires voice of the patient, and pain may be recognized by recognizing that the patient has complained of pain from the voice. An output signal of the communication device 510, a facial expression recognition result, or a voice recognition result is input to the situation recognition section 120 as input pain information INPN.

The image acquisition section 141 receives an endoscopic image transmitted by the signal processing device 310 of the endoscope device 300, thereby acquiring the endoscopic image. The endoscope shape acquisition section 142 acquires the position and the shape of the endoscope insertion section based on the detection signal from the endoscope shape acquisition sensor 20. The endoscopic image and information of the position and shape of the endoscope insertion section are input to the situation recognition section 120 as the information IFIN.

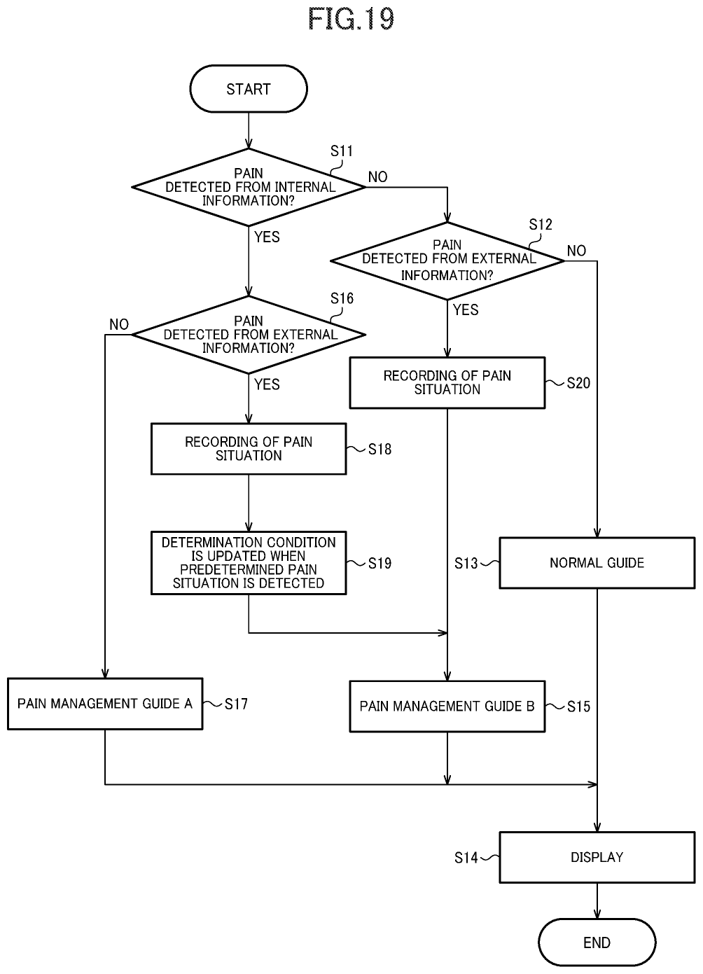

FIG. 19 is a flowchart of the processing performed by the insertion support system 100 in the second detailed configuration example. In this flowchart, the insertion support information generated in the steps S13 and S17 corresponds to the insertion support information generated when the input pain information INPN is not input described in FIG. 1. Further, the insertion support information generated in the step S15 corresponds to the insertion support information generated when the input pain information INPN is input described in FIG. 1.

In the step S11, the insertion support information generation section 150 determines whether or not the pain situation recognition section 130 has detected a pain situation based on internal information. The internal information refers to information other than the input pain information INPN transmitted by the patient or a medical worker. In the example of FIG. 18, the internal information is the endoscopic image acquired by the image acquisition section 141, the insertion section shape information acquired by the endoscope shape acquisition section 142, the latest status information TYK output by the latest status determination section 121, the time-series status information JIK output by the time-series status determination section 122, or any combination of two or more of them.

When the pain situation recognition section 130 detects a pain situation based on the internal information in the step S11, in the step S16, the insertion support information generation section 150 determines whether or not the pain situation recognition section 130 detects the pain situation based on external information. The external information is the input pain information INPN transmitted by the patient or a medical worker.

In the step S16, when the pain situation recognition section 130 does not detect a pain situation based on the external information, the insertion support information generation section 150 generates insertion support information of a pain management guide A. The pain management guide A is used to guide the operations for each of the pain situations (1) to (23) described above.

When the pain situation recognition section 130 detects a pain situation based on the external information in the step S16, in the step S18, the pain situation recognition section 130 records the detected pain situation in the memory as a log. In the step S19, when the detected pain situation corresponds to any of the predetermined pain situations (1) to (23) described above, the pain situation recognition section 130 updates the determination conditions for the corresponding pain situation. For example, if a pain situation is detected by threshold determination of the displacement amount, the threshold is updated. In the step S15, the insertion support information generation section 150 generates insertion support information of a pain management guide B. The pain management guide B corresponds to each of the pain situations (1) to (23) described above, and also guides operations in consideration of the pain specific to the patient. The pain specific to the patient refers to a pain caused under an insertion status different from the pain situations (1) to (23). The operation in consideration of the pain specific to the patient is an operation for avoiding a pain situation specific to the patient. In the step S14, the insertion support information generation section 150 displays the generated insertion support information in the display device 220.

When the pain situation recognition section 130 does not detect a pain situation based on the internal information in the step S11, in the step S12, the insertion support information generation section 150 determines whether or not the pain situation recognition section 130 detects the pain situation based on the external information.

In the step S12, when the pain situation recognition section 130 does not detect a pain situation based on the external information, in the step S13, the insertion support information generation section 150 generates insertion support information of a normal guide corresponding to the current insertion status. The normal guide is used to guide operations when pain is not occurring. In the step S14, the insertion support information generation section 150 displays the generated insertion support information in the display device 220.

When the pain situation recognition section 130 detects a pain situation based on the external information in the step S12, in the step S20, the pain situation is recorded. For example, the pain situation is recorded in electronic health records, or the like. When the pain situation recognition section 130 recognizes a pain situation that does not fall into any of the pain situations (1) to (23) described above based on the external information, by recording the pain situation as a pain specific to the patient, when a similar pain situation occurs again in the current examination or in another examination performed in the future, such a pain specific to the patient can be recognized. Next, in the step S15, the insertion support information generation section 150 generates insertion support information of a pain management guide B. In the step S14, the insertion support information generation section 150 displays the generated insertion support information in the display device 220.

### 5. Third and Fourth Detailed Configuration Examples of Insertion Support System

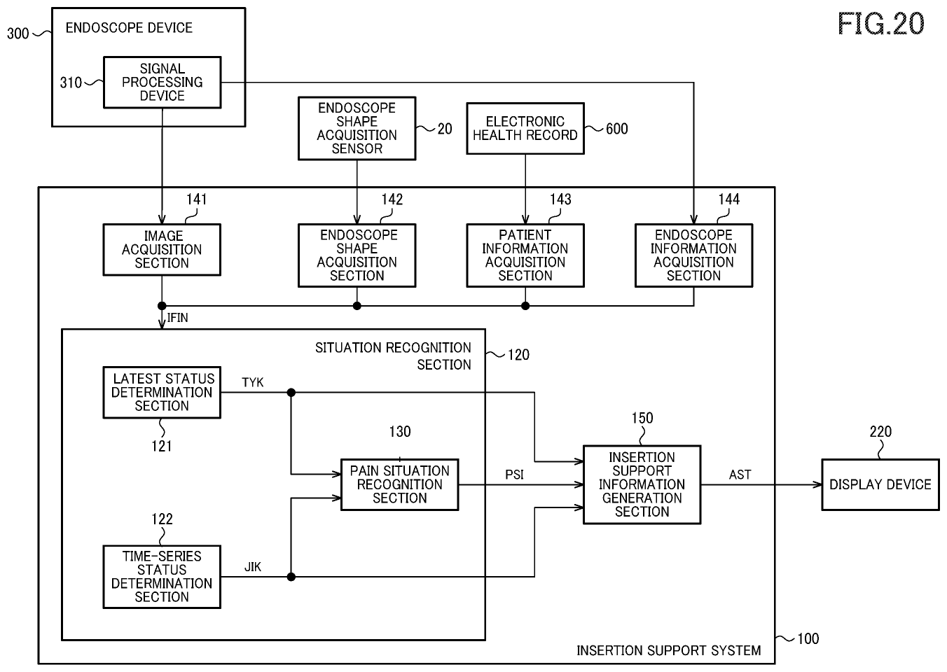

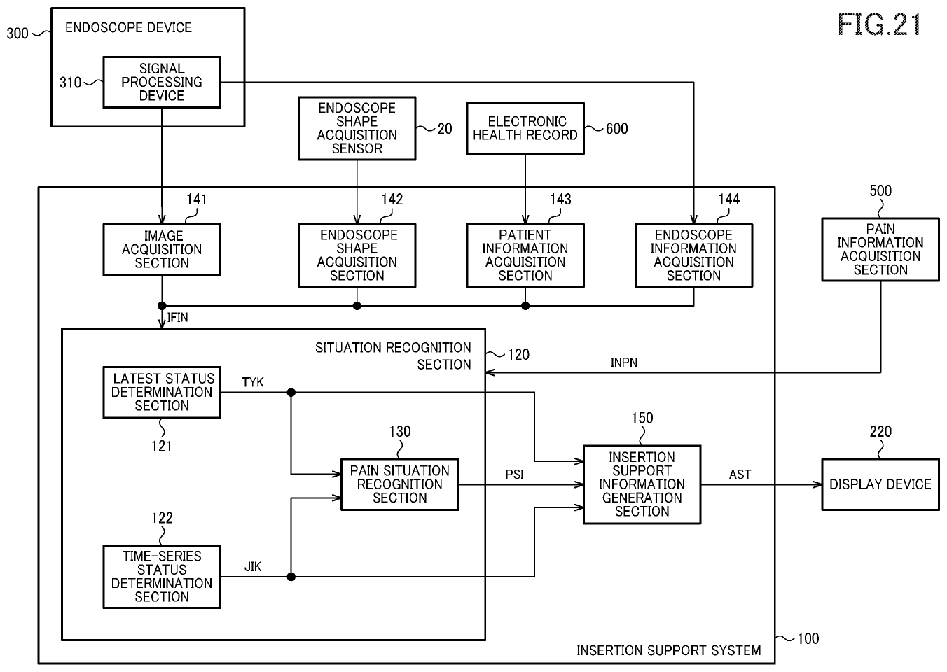

FIG. 20 is a third detailed configuration example of the insertion support system 100. The insertion support system 100 includes the image acquisition section 141, the endoscope shape acquisition section 142, the patient information acquisition section 143, the endoscope information acquisition section 144, the situation recognition section 120, and the insertion support information generation section 150. FIG. 21 is a fourth detailed configuration example of the insertion support system 100. The fourth detailed configuration example has a configuration in which the pain information acquisition section 500 is further added to the third detailed configuration example. In the third and fourth configuration examples, for example, the image acquisition section 141, the endoscope shape acquisition section 142, and the latest status determination section 121 correspond to the endoscopy status information acquisition section 110 in FIGS. 1 and 2, and the patient information acquisition section 143, the endoscope information acquisition section 144, and the time-series status determination section 122 correspond to the examination condition information acquisition section 160 in FIG. 2. The same components as those already described will be omitted from the description as appropriate.

An electronic health record 600 accumulates patient information, which is information about patient attributes. The electronic health record 600 is stored in, for example, a storage device provided outside the endoscope system 400, and the patient information acquisition section 143 acquires the electronic health record 600 from that storage device.

The patient information is, for example, the physique, gender, age, medical history, body fat percentage of the patient, or any combination of two or more of them. The physique is BMI, height, weight, or any combination of two or more of them. Depending on these patient attributes, the pain situations that are likely to occur, or the pain thresholds, may differ. For example, a thin woman may feel pain easily, and men feel pain when the N-loop is extended 50 mm to the head side. By recognizing a pain situation based on the patient information, the pain situation specific to the patient can be appropriately recognized. Therefore, appropriate insertion support can be achieved.

Further, the patient information may also include past examination information. The past examination information refers to information as to which insertion method was used, how many minutes it took to insert, whether or not pain occurred, the trajectory of the insertion, which scope was used, whether or not sedatives were used, or any combination of two or more of these information items. By recognizing a pain situation based on the past examination information, the pain situation specific to the patient can be appropriately recognized. Therefore, appropriate insertion support can be achieved.

The endoscope information acquisition section 144 acquires endoscope kind information from the signal processing device 310 of the endoscope device 300. The thickness of the endoscope insertion section varies depending on the kind of endoscope. Therefore, the pain that is likely to occur may vary depending on the kind of endoscope. Therefore, when the pain situation is recognized based on the endoscope kind information, different pain situations are appropriately recognized depending on the kind of endoscope. Therefore, appropriate insertion support can be achieved.

### 6. Fifth Detailed Configuration Example of Insertion Support System

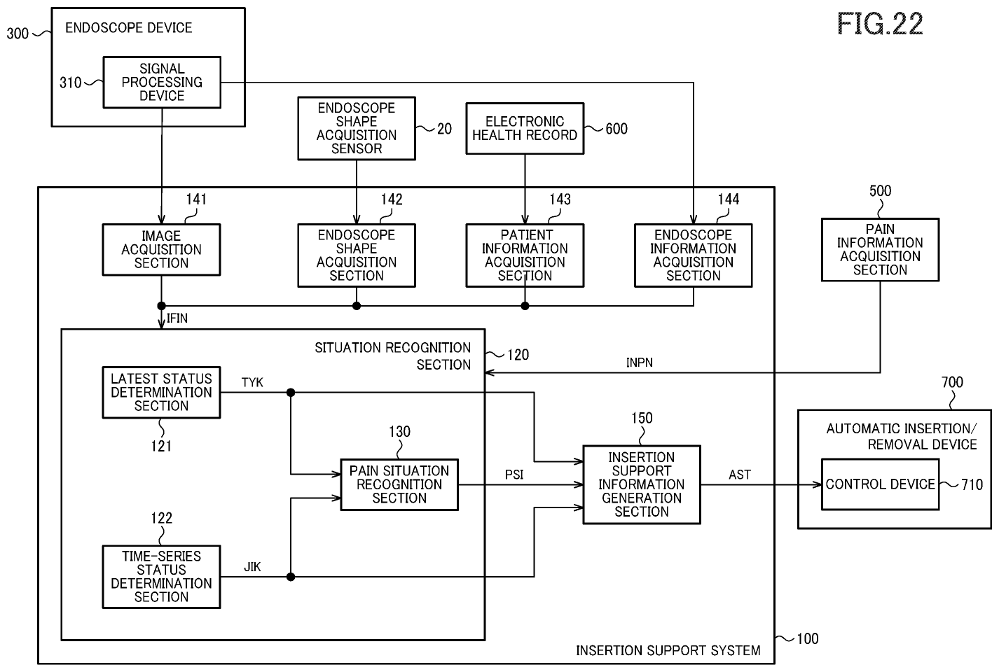

FIG. 22 is a fifth detailed configuration example of the insertion support system 100. The insertion support system 100 includes the image acquisition section 141, the endoscope shape acquisition section 142, the patient information acquisition section 143, the endoscope information acquisition section 144, the situation recognition section 120, and the insertion support information generation section 150. The same components as those already described will be omitted from the description as appropriate.

The insertion support information generation section 150 may control an automatic insertion/removal device 700 by outputting support information AST to a control device 710 of the automatic insertion/removal device 700. The automatic insertion/removal device 700 is a robot that inserts and removes an endoscope automatically or semi-automatically, and the control device 710 is a device for controlling the robot. The support information AST is output as a control signal to the control device 710. When the pain situation recognition section 130 recognizes a pain situation, the insertion support information generation section 150 may, for example, output a control signal to stop the operation of the automatic insertion/removal device 700, or output a control signal to change the operation to an operation different from the operation when the pain situation is not recognized.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. An insertion support system comprising:
a processor,
wherein the processor is configured to:
acquire endoscopy status information associated with an endoscopy using an endoscope, the endoscopy status information including at least one of an endoscopic image, insertion section shape information, or operation recognition information, the insertion section shape information being information about a shape of an endoscope insertion section, the operation recognition information being information about a change in at least one of the shape or a position of the endoscope insertion section;
receive input of at least one of input pain information or the endoscopy status information, the input pain information being information about a pain of a patient acquired from the patient or a medical worker in real time, and acquire pain situation information upon recognition of a pain situation, the pain situation being a situation where the patient is suffering a pain during the endoscopy;
acquire examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and
generate insertion support information according to the pain situation information, the endoscopy status information, and the examination condition information.

2. The insertion support system as defined in claim 1, wherein
when both of the input pain information and the endoscopy status information are input, the processor recognizes the pain situation based on the input pain information.

3. The insertion support system as defined in claim 2, wherein
when the endoscopy status information is input while the input pain information is not input, the processor recognizes the pain situation based on the endoscopy status information, and generates the insertion support information according to the pain situation recognized based on the endoscopy status information, and
when the input pain information is input in addition to the endoscopy status information, the processor recognizes the pain situation based on the input pain information, and generates the insertion support information according to the pain situation recognized based on the input pain information.

4. The insertion support system as defined in claim 1, wherein
the processor
classifies an insertion status of the endoscope based on at least one of the endoscopic image, the insertion section shape information, and the operation recognition information, and outputs the endoscopy status information including classification results, and
recognizes whether or not the insertion status indicated by the classification results corresponds to the situation where the patient is suffering a pain, thereby recognizing the pain situation.

5. The insertion support system as defined in claim 4, wherein
the operation recognition information includes insertion section shape displacement information being information about shape displacement of the endoscope insertion section, and
the processor performs the classification based on the insertion section shape information and the insertion section shape displacement information.

6. The insertion support system as defined in claim 5, wherein
the insertion section shape displacement information includes shape displacement amount information being information about magnitude of the shape displacement, and
the processor performs the classification based on the insertion section shape information and the shape displacement amount information.

7. The insertion support system as defined in claim 1, wherein
when the processor generates the insertion support information corresponding to the insertion status of the endoscope based on the endoscopy status information, the processor generates first insertion support information as the insertion support information when the pain situation information not indicating the situation where the patient is suffering a pain is input, and generates second insertion support information being different from the first insertion support information, as the insertion support information when the pain situation information indicating the situation where the patient is suffering a pain is input.

8. The insertion support system as defined in claim 1, wherein
the processor recognizes the pain situation by recognizing, based on the endoscopy status information, a situation where the pain is caused by extension of an intestinal wall of a large intestine, a situation where a pain is caused by a mesentery being pulled, a situation where the pain is caused by the endoscope insertion section pushing the intestinal wall, or a situation where the pain is caused due to a relationship between a fixed colon of the large intestine and the endoscope insertion section.

9. The insertion support system as defined in claim 1, wherein
the processor recognizes the pain situation where the patient is suffering a pain when the processor recognizes, based on the endoscopy status information, a situation where a push up operation to a flank side or a head side is performed near SDJ by an angle operation of the endoscope.

10. The insertion support system as defined in claim 1, wherein
the processor recognizes the pain situation where the patient is suffering a pain when the processor recognizes, based on the endoscopy status information, a situation where a push up operation to a head side is performed near SDJ by a torque operation and an angle operation of the endoscope.

11. The insertion support system as defined in claim 1, wherein
the processor recognizes the pain situation where the patient is suffering a pain when the processor recognizes, based on the endoscopy status information, a situation where an operation is performed near SDJ with the endoscope insertion section being bent.

12. The insertion support system as defined in claim 1, wherein
the processor recognizes the pain situation where the patient is suffering a pain when the processor recognizes, based on the endoscopy status information, a situation where a pull operation is performed with looping of the endoscope.

13. The insertion support system as defined in claim 1, wherein
the processor recognizes the pain situation where the patient is suffering a pain when the processor recognizes, based on the endoscopy status information, a situation where a distal end of the endoscope insertion section is present in any of a splenic flexure, a transverse colon, and a hepatic flexure, and a push operation is performed when a re-loop is formed in a sigmoid colon.

14. The insertion support system as defined in claim 1, wherein
the processor recognizes the pain situation where the patient is suffering a pain when the processor recognizes, based on the endoscopy status information, a situation where a distal end of the endoscope insertion section is present in any of a splenic flexure, a transverse colon, and a hepatic flexure, and a pull operation is performed upon dealing with a re-loop in a sigmoid colon.

15. The insertion support system as defined in claim 1, wherein
the processor recognizes the pain situation where the patient is suffering a pain when the processor recognizes, based on the endoscopy status information, a situation where a left colon portion is pushed up from a splenic flexure by a push operation of the endoscope.

16. The insertion support system as defined in claim 1, wherein
the input pain information is at least one of patient facial expression information being information about patient's facial expression, and pain communication information being information from a communication device operated by the patient or a medical worker, during the endoscopy.

17. The insertion support system as defined in claim 1, wherein
the processor recognizes the pain situation by distinguishing situations with different pain levels, different pain frequencies, or different degrees of pain avoidability from each other.

18. The insertion support system as defined in claim 1, wherein
the processor
acquires examination condition information including at least one of endoscope kind information, patient information, and past examination information, the endoscope kind information being information about a kind of the endoscope insertion section used for the endoscopy, the patient information being information about the patient attributes, and the past examination information being information about past endoscopies, and
recognizes the pain situation based on the examination condition information.

19. An endoscope system, comprising:
an endoscope for use in endoscopy; and
a processor,
wherein the processor is configured to:
acquire endoscopy status information associated with the endoscopy using the endoscope, the endoscopy status information including at least one of an endoscopic image, insertion section shape information, or operation recognition information, the insertion section shape information being a shape of an endoscope insertion section, the operation recognition information being information about a change in at least one of the shape or a position of the endoscope insertion section;
receive input of at least one of input pain information or the endoscopy status information, the input pain information being information about a pain of a patient acquired from the patient or a medical worker in real time, and acquire pain situation information upon recognition of a pain situation, the pain situation being a situation where the patient is suffering a pain during the endoscopy;
acquire examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and
generate insertion support information according to the pain situation information, the endoscopy status information, and the examination condition information.

20. An insertion support method, comprising:
acquiring endoscopy status information associated with an endoscopy using an endoscope, the endoscopy status information including at least one of an endoscopic image, insertion section shape information, or operation recognition information, the insertion section shape information being a shape of an endoscope insertion section, the operation recognition information being information about a change in at least one of the shape or a position of the endoscope insertion section;
receiving input of at least one of input pain information or the endoscopy status information, the input pain information being information about a pain of a patient acquired from the patient or a medical worker in real time, and acquiring pain situation information upon recognition of a pain situation, the pain situation being a situation where the patient is suffering a pain during the endoscopy;
acquiring examination condition information including at least one of endoscope kind information, patient information, and past examination information, the endoscope kind information being information about a kind of the endoscope insertion section used for the endoscopy, the patient information being information about patient attributes, and the past examination information being information about past endoscopies; and
generating insertion support information according to the pain situation information, the endoscopy status information, and the examination condition information.
